(19) United States
(12) Patent Application Publication
(10) Pub. No.: US 2005/0131249 A1
(43) Pub. Date: Jun. 16, 2005

(54) Method of making salt

(75) Inventors: David George Stedman, Benoni (ZA); Robert Fletcher Kenyon, Surrey (GB)

Correspondence Address: HOWREY SIMON ARNOLD & WHITE LLP, c/o IP DOCKETING DEPARTMENT, 2941 FAIRVIEW PARK DRIVE, SUITE 200, FALLS CHURCH, VA, 22042-7195, US

(73) Assignee: Technical and Commerical Services International

(21) Appl. No.: 10/511,004

(22) PCT Filed: May 8, 2002
(86) PCT No.: PCT/IB02/01554

(30) Foreign Application Priority Data
Apr. 12, 2002 (ZA) 2002/2902

(51) Int. Cl. C07C 53/134; C07F 9/22

(52) U.S. Cl. 562/8; 562/496

(57) ABSTRACT

A method of making the salt of an acid, includes the steps of combining and mixing the acid and a base selected from the oxides, hydroxides and carbonates of sodium, potassium, calcium and magnesium, in a first reaction zone over a first period to produce a reaction mixture. The reaction mixture is transferred at the end of the first period from the first reaction zone to a second reaction zone the transferring step being carried out over a second period. Heat generated by reaction between the acid and the base in the second reaction zone drives off sufficient water to produce a product mixture containing less than about 8% (m/m) water. The first period is between about 1 and 180 seconds and the second period is between about 2 and 60 seconds.

THIS INVENTION relates to a method of making a salt.

The metal salts of lower molecular mass acids such as substituted and unsubstituted C₁-C₁₀ acids, aromatic carboxylic acids of the formula Ph-(CH₂)ₓ—CO₂H where x is 0-4, benzoic acid and phenylacetic acid are typically made by reacting the acid with a basic salt of the metal such as its hydroxide or carbonate. In some cases, the reactions are conducted in a relatively dilute aqueous medium and isolation of the salt of the acid requires removal of excess water and a drying step. In other cases, the acid is added to a slurry of a base such as calcium carbonate or calcium hydroxide in a closed vessel. The slurry then progressively thickens and passes through a “plastic” stage after which it is dried. Handling the product after the plastic stage is generally difficult. Furthermore large scale production process drying is energy intensive and expensive. It is an object of the invention to address these problems.

According to a first aspect of the invention, there is provided a method of making the salt of an acid selected from C₁-C₁₀ carboxylic acids, aromatic carboxylic acids of the formula Ph-(CH₂)ₓ—CO₂H where x is 0-4, and glycerophosphoric acid, the method including the steps of

combining and mixing the acid and a base selected from the oxides, hydroxides and carbonates of sodium, potassium, calcium and magnesium, or a mixture of any two or more thereof, in a first reaction zone, the combining and mixing step being carried out over a first period to produce a reaction mixture in the first reaction zone;

transferring the reaction mixture at the end of the first period from the first reaction zone to a second reaction zone the transferring step being carried out over a second period; and

allowing heat generated by reaction between the acid and the base in the second reaction zone to drive off sufficient water to produce a product mixture containing less than about 8% (m/m) water.

The first period may be 1-180 seconds and the second period may be 2-60 seconds. More particularly, the first period may be 3-45 seconds and the second period may be 2-20 seconds.

The combining step may take about 2-60 seconds and preferably takes about 3-15 seconds.

The first period, i.e. the total time for combining and mixing the acid and the base prior to commencing the transfer step, will preferably be about 3-60 seconds. The second period will preferably be about 3-30 seconds.

The carboxylic acid may be a substituted or an unsubstituted C₁-C₁₀ acid. It may be a monocarboxylic acid or a dicarboxylic acid. The monocarboxylic acid may be selected from formic acid, acetic acid, propanoic acid, butanoic acid, pentanoic acid, hexanoic acid, heptanoic acid, octanoic acid, nonanoic acid, decanoic acid, benzoic acid and their alkylated or hydroxylated analogues. The dicarboxylic acid may be selected from oxalic acid, malonic acid, succinic acid, glutaric acid, adipic acid, pimelic acid, suberic acid, azelaic acid, sibaccic acid and their unsaturated, alkylated or hydroxylated analogues such as fumaric acid, malic acid and citric acid. The carboxylic acid may be benzoic or phenylacetic acid. The carboxylic acid may include at least one heteroatom. However, the invention is not limited to these acids.

The method may include adding further quantities of the acid (either concentrated or diluted) to make up for evaporative loss of acid. Typically this not only boosts the acid content and moisture of the product but also encourages a certain amount of agglomeration or granulation of the product. This may require additional drying as a result of the water added or produced.

The term “% (m/m)” refers to a mass per mass percentage.

The acid may be in the form of an aqueous solution. The aqueous solution may have a concentration of about 60-100% by mass of the acid. Preferably, it will have a concentration of about 50-99,5% by mass of the acid. The method may include the prior step of mixing the acid and water. The acid and the water may thus be kept in separate storage tanks.

The amount of water used will be determined by the reactivity of the acid and the base. For example, if lower molecular mass and more volatile acids such as acetic acid or propanoic acid are reacted with a base such as calcium oxide in the absence of water, the temperature can rise to over 170° C. This can cause charring and the loss of up to 25% of the acid by evaporation. Overheating is avoided by using an aqueous solution of the acid at a concentration of about 65-97%. In this way, charring is avoided and evaporative loss is greatly reduced. The acid may be diluted beforehand or acid and water may be added simultaneously to a reaction vessel.

The choice of base is also determined by the reactivity of the acid. Generally the purity of commercial acids and bases is about 80-99%. Again, in the case of the lower molecular mass more reactive acids, a less reactive base such as calcium carbonate or calcium hydroxide, or a mixture of these with calcium oxide, may be used to limit or avoid overheating. The bases may be premixed or added simultaneously to a reaction vessel.

In the case of the lower molecular mass and more volatile acids, an excess of about 5-10% above the stoichiometric amount may be used to allow for evaporative loss.

The reaction between the acid and the base takes place rapidly with the evolution of a large amount of heat. Water evaporates and the product thickens to produce a magma.

Preferably, the method will include allowing the heat generated to drive off sufficient water to produce a product mixture containing less than about 8% water and more preferably less than about 2,5% water.

The combining step may include adding the base to a solution of the acid. The method may include the prior step of warming the solution of acid to about 50-96° C. and preferably to about 75-85° C.

The duration of the combining and mixing step, ie the duration of the first period, will vary with the amounts of the acid and base and with the nature of the base. The duration of the second period will vary similarly. The outlet of the mixing vessel will be large to facilitate rapid discharge of the magma at the end of the first period.

The method may include the further steps of successively combining and mixing a plurality of batches of the acid, base and water in the first reaction zone to produce successive batches of the reaction mixture and successively transferring each of the batches to the same second reaction zone.

The second reaction zone may be a receiving vessel and several batches of reaction mixture may be transferred into the same receiving vessel until the receiving vessel is full or contains a predetermined quantity of the reaction mixture. The receiving vessel may then be replaced with a second receiving vessel and further batches may be added to the second receiving vessel. This process may be continued until the initially used receiving vessels have been emptied so that they can be reused. It is an advantage of this embodiment of the invention that the reaction vessel and all of the receiving vessels can be kept within an enclosed area so that steam and fumes produced in the reaction between the acid and the calcium salt can be extracted and dealt with by an appropriate disposal system.

In an example of this embodiment of the invention, a material dosing system, controlled by a programmable logic controller or similar suitable control unit, weighs out the raw materials in sequence into a mixing vessel. An agitator in the mixing vessel starts immediately on receiving a signal that the first of the acid has been dosed. Optionally there can be an operator-controlled delay between the opening of the acid dosing valves and the beginning of the agitation.

Thus the method may be one in which a material dosing system, controlled by a programmable logic controller, weighs out the raw materials into the mixing vessel/first reaction zone.

Once agitation has begun, the base is dosed. The raw materials are then mixed for a specific time before being discharged into a receiving vessel. Additional batches are then dosed into the same reaction vessel and discharged into the receiving vessel until the amount of material in the receiving vessel has reached a desired level. Optionally, the material in the receiving vessel can also be agitated. Agitation of the reaction mixture in the receiving vessel facilitates the liberation of steam arising from the reaction and aids in drying the product. Once the desired level in the receiving vessel has been reached, it is removed from the dosing area and replaced by a fresh receiving vessel. The process then continues with reaction mixtures being charged into the fresh receiving vessel. The first receiving vessel can then be moved to another location whilst the reaction proceeds to completion with the heat produced driving off water. Instead, only a portion of the contents of the first receiving vessel may be discharged, the balance being retained in the first receiving vessel into which fresh mixture from the reaction vessel is discharged to be blended with earlier-made product. Once the material in the first receiving vessel is dry enough, the contents may be discharged and the vessel reused. The contents of the first receiving vessel are generally discharged when the second receiving vessel is full and the contents of the second vessel are discharged when the first vessel is full. The discharged material is allowed to cool with occasional agitation to allow heat to escape. The final product is then dressed according to size and packaged.

In a particular embodiment of the invention, the raw materials were individually weighed in batches from weigh hoppers into an agitated mixing vessel. Fumes from the mixing vessel were withdrawn by an extraction system. After the prescribed mixing, or first, period the contents of the mixing vessel were rapidly discharged into a second mixing vessel where the reaction was allowed to proceed. The fumes produced in the second mixing vessel were also withdrawn by an extraction system. The second mixing vessel was agitated to encourage the liberation of steam produced by the reaction and to aid in drying the product. Successive batches were then discharged from the initial mixing vessel into the second mixing vessel until it was full. Either all or a portion of the product was then discharged and the procedure was started again. The product produced was removed for further processing. In another embodiment of a continuous operation, the raw materials were individually weighed in batches from weigh hoppers into an agitated mixing vessel. Fumes from the mixing vessel were withdrawn by an extraction system as before. After the prescribed mixing, or first, period the contents of the mixing vessel were rapidly discharged into a second mixing vessel where the reaction was allowed to proceed. The fumes produced in the second mixing vessel were again withdrawn by an extraction system. However, only a portion of the product was discharged, either continuously or on a semi-continuous basis, the balance remaining in the second mixing vessel. Accordingly, at all times, there was fresh material reacting whilst being blended in with material already produced. In different embodiments, the individual materials were either individually weighed or were pre-blended where this was suitable.

There is essentially no limit to the number of receiving vessels that can be employed and the size of the reaction vessels can be selected to suit the residence time required to ensure complete reaction and a desired output.

Preferably, the reaction vessels will be designed to have no sharp internal corners or edges. The vessels will also preferably be lined with an inert material such as a layer of PTFE (polytetrafluoroethylene) or a similar material. The top of the mixing/reaction vessel will preferably be provided with an extraction system for extraction of steam and vapour. The inner lining material serves to reduce adhesion between the reactants, the product and the vessel to facilitate rapid discharge when the vessel is emptied.

According to a second aspect of the invention, there is provided a method of making the salt of an acid selected from C₁-C₁₀ carboxylic acids, aromatic carboxylic acids of the formula Ph-(CH₂)ₓ—CO₂H where x is 0-4, and glycerophosphoric acid, the method including the steps of

combining and mixing the acid, a base selected from the oxides, hydroxides and carbonates of sodium, potassium, calcium and magnesium, or a mixture of any two or more thereof and water in a first reaction zone to produce a reaction mixture in the first reaction zone;

continuously transferring the reaction mixture from the first reaction zone to a second reaction zone, the reactants being added to the first reaction zone in successive batches and the reaction mixture being continuously removed from the first reaction zone at a rate which is selected so that the residence time of the reaction mixture in the first reaction zone is between about 1 and 180 seconds; and

allowing heat generated by reaction between the acid and the base in the second reaction zone to drive off sufficient water to produce a product mixture containing less than about 8% water,

provided that the acid is not 2-hydroxy-4-thiomethylbutyric acid.

Preferably the method will involve allowing the heat generated to drive off sufficient water to produce a product mixture containing less than about 2,5% water.

The rate of removal of the reaction mixture from the first reaction zone will depend on the production capacity of the installation and may vary between about 100 kg and about 30,000 kg per hour.

According to another aspect of the invention, there is provided a continuous method of making the salt of an acid selected from C₁-C₁₀ carboxylic acids, aromatic carboxylic acids of the formula Ph-(CH₂)ₓ—CO₂H where x is 0-4, and glycerophosphoric acid, the method including the steps of

simultaneously feeding, into a reaction zone, an aqueous solution of the acid, and a base selected from the oxides, hydroxides and carbonates of sodium, potassium, calcium, magnesium, or a mixture of any two or more thereof to produce a reaction mixture in the reaction zone; and

transferring the reaction mixture from the reaction zone to a second zone at a rate which is selected so that the residence time of the reaction mixture in the reaction zone is sufficient to allow heat generated by reaction between the acid and the base in the reaction zone to drive off sufficient water to produce a product mixture containing less than about 8% water,

provided that the acid is not 2-hydroxy-4-thiomethylbutyric acid.

The rate will preferably be selected so that the residence time of the reaction mixture in the reaction zone is about 3-20 minutes.

According to another aspect of the invention, there is provided a continuous method of making a salt of an acid selected from C₁-C₁₀ carboxylic acids, aromatic carboxylic acids of the formula Ph-(CH₂)ₓ—CO₂H where x is 0-4, and glycerophosphoric acid, the method including the steps of

simultaneously feeding, into a reaction zone, an aqueous solution of the acid, and a base selected from the oxides, hydroxides and carbonates of sodium, potassium, calcium and magnesium, or a mixture of any two or more thereof to produce a reaction mixture in the reaction zone; and

transferring the reaction mixture from the reaction zone to a second zone at a rate which is selected so that the residence time of the reaction mixture in the reaction zone is sufficient to initiate reaction between the acid and the base but not sufficient to drive off water from the reaction mixture, and allowing heat generated by further reaction between the acid and the base in the second zone to drive off sufficient water to produce a product mixture containing less than about 8% water, provided that the acid is not 2-hydroxy-4-thiomethylbutyric acid.

The rate of addition of the acid and the salt will depend upon the production capacity of the plant in which the process is conducted. Production levels of 100-30,000 kg per hour can be achieved by the method of the invention.

In an example of this embodiment of the invention, the amount of material retained in the mixing chamber is less and the mixing chamber serves only to mix the two reactants and to allow reaction to initiate. In this case the residence time in the mixing zone is about 3-180 seconds. Further reaction and water loss from the reaction mixture then continues after the mixture has been discharged from the mixing vessel. The product is then screened or packaged as described above.

The reaction mixture in the mixing/reaction zone may be transferred on an endless moving belt so that the second zone is formed by the belt. The reactants will then react whilst being carried on the belt. The length of the belt and the speed of the belt will be selected so that, when the material is discharged from the belt, the resulting product mixture contains the desired amount of water. The belt will preferably be enclosed so that steam and fumes produced by reaction between the acid and the calcium salt can be extracted and dealt with by an appropriate disposal system as described above.

Preferably, at the point of discharge from the belt, the material will be passed through a rotating cutter, to reduce the particle size and to release trapped steam and gases in the product. The product will then be transported on a conveyor or suitable handling device for further processing as required, for example for granulation, drying, cooling, dressing or bagging.

There are several important advantages associated with this embodiment of the invention. Because there is minimal mixing of the product after the initial feeding and mixing stage, energy requirements of the process are reduced. Furthermore, when the product passes through the plastic stage, no mechanical agitation takes place. There is therefore no contact between a mixing device and the thickening product and no build-up or aggregation of material in the mixing vessel or on the agitator. It has been found that this largely removes the requirement of high pressure steam cleaning of equipment and the resulting effluent problem. It is also an advantage that the entire reaction system is enclosed. This allows relatively easy removal of steam, water vapour and gases produced in the reaction.

In an example of this embodiment of the invention, the aqueous acid solution is weighed out into the mixing/reaction vessel. The agitator is started and the base is dosed. After a predetermined period, the outlet valve of the mixing vessel opens and the contents discharge onto an endless enclosed belt running, typically, between two pulleys.

In this embodiment of the invention, an agitator can be included at the beginning of the belt close to the discharge from the mixing/reaction vessel to ensure that complete mixing takes place before the material passes from the fluid stage. Preferably, the initial part of the belt has a U-shaped cross-section in order to hold the relatively fluid reaction mixture. The initial part of the belt is also arranged to slope downwardly from the reaction vessel to prevent reverse flow or spillage of material. The U-shaped section is selected to have a volume which is sufficient to hold up to 30 minutes of plant production capacity. During this period, the reaction mixture passes through a fluid and then a plastic stage with the evolution of steam and gases. At the end of the U-shaped section, the belt flattens out to almost its full width but remains slightly curled at the outside edges to minimize the risk of spillage. At the point at which the belt flattens out, the product has already partially dried. The flattening of the belt causes the cake to split open to release steam, moisture and gases trapped inside the cake. The thickness of the cake on the belt generally varies between about 5 and about 90 cm. The cake is then carried by the belt, drying as it moves and, at the end of the belt, the cake falls into a crumbling device. This reduces the particle size of the product and releases steam, moisture and trapped gases. The product is then transported to a second locality for treatment such as granulation, drying, cooling, enrichment, sizing or bagging.

In a preferred embodiment, the entire belt is enclosed by a canopy. Air is drawn through the enclosed space carrying with it steam, water vapour, fumes and gases produced in the reaction. The extracted gases are treated in a suitable treatment plant.

Preferably the belt will be made of a suitable high temperature resistant material, PTFE, thin stainless steel, wooden slats or the like. The release-nature of the belt is also important. The belt must be sufficiently smooth to allow the product to drop off the belt as it passes around the final pulley. Ideally, no material should adhere to the belt. The enclosed belt system will typically have a width of up to about 2 m, a length of approximately 25 m and a maximum height of about 2 m.

The invention extends to a calcium, magnesium, sodium or potassium salt of C₁-C₁₀ acid, aromatic carboxylic acids of the formula Ph-(CH₂)ₓ—CO₂H where x is 0-4, or of glycerophosphoric acid prepared by a method as hereinbefore described.

Where the raw materials, or reactants, are dosed on a batch basis, the dosage can be by weight or volume. Where the reactants are dosed on a continuous basis, the amount dosed will be measured by a belt weigher, mass flow meter or a similar dosing device.

The invention is now described, by way of example, with reference to the following Examples and the drawings in which

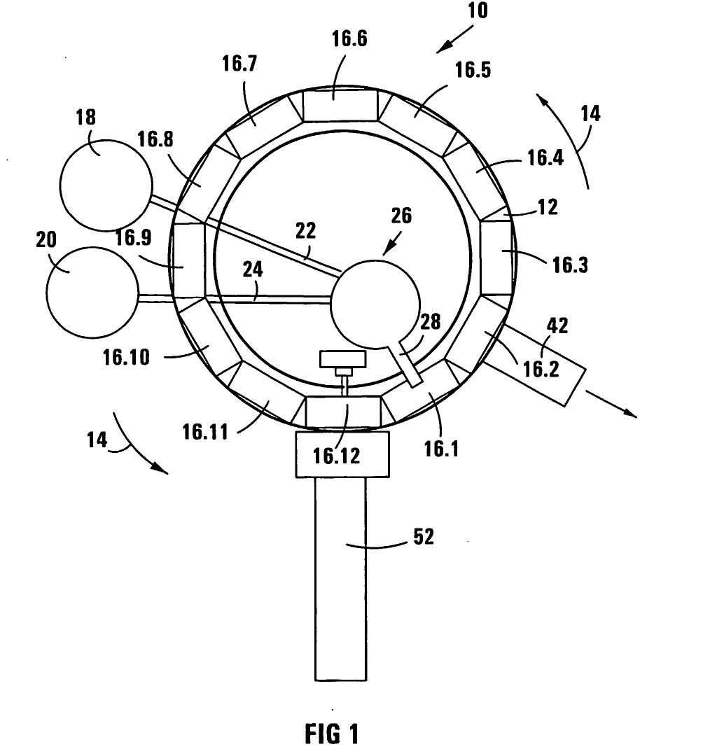

FIG. 1 shows a schematic plan view of an installation for use in the method of the invention;

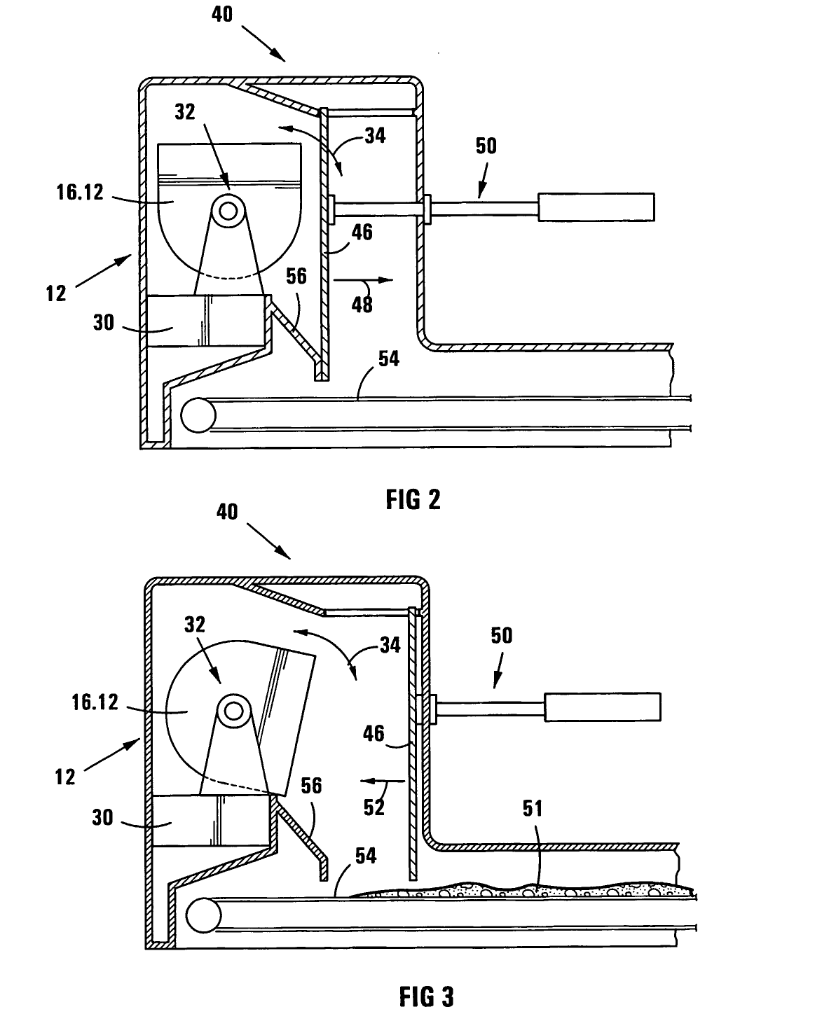

FIG. 2 shows a schematic side view of part of the installation of FIG. 1 with a hopper in an upright position;

FIG. 3 shows the side view of FIG. 2 with the hopper in a tilted position; and

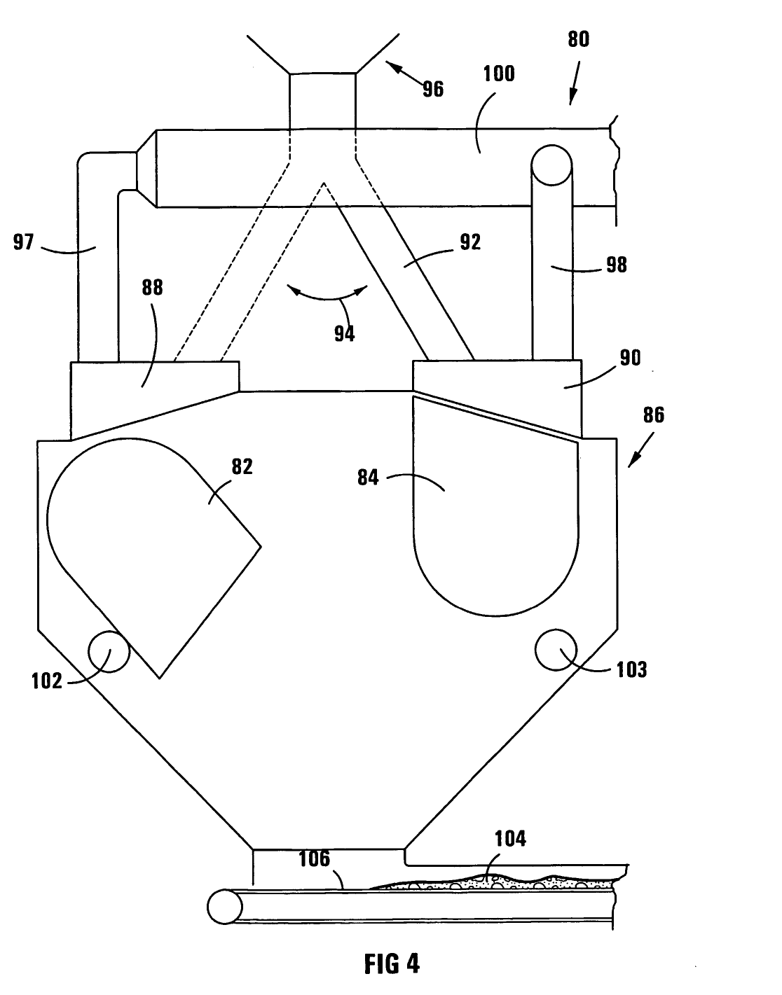

FIG. 4 shows a schematic side view of another installation for use in the method of the invention.

### EXAMPLE 1

### Calcium Propanoate

Five samples of commercial propionic acid (totalling 817,5 g) were weighed out into five containers. In each case, the mass was 5% in excess of the stoichiometric requirement. The acid temperature and ambient temperature were 26° C. Five samples of commercially available quicklime (calcium oxide) (totally 293 g) were also pre-weighed.

Acid from a first container was rapidly discharged into a mixing vessel which had a volume of approximately 8 times the total volume of the reactants, over a period of about two to three seconds. The entire amount of the oxide was immediately added to the acid with rapid agitation.

The reaction started almost immediately and was characterized by a rapid increase in the temperature of the solution and the evolution of fumes. After 15 seconds of vigorous mixing, the magma was discharged into a separate container. The temperature of the magma rose to 110° C. within 60 seconds of commencement of the acid addition.

The remaining four samples of acid and of oxide were added sequentially to the mixing vessel in the order acid and then oxide. In each case the same sequence of addition of acid and addition of oxide followed by stirring and discharge was followed. Each charge took a maximum of 30 seconds from the start of the acid addition to the mixing container being completely emptied.

By the end of the addition phase, the magma temperature had risen to 117° C. The temperature continued to rise to a peak of 170° C. approximately 18 minutes after the initial acid batching. The magma dried and formed a mixture of flakes and powder approximately 6 minutes after commencement of the trial.

As the temperature of the magma approached 155° C. some of the material pushed up by steam pockets in the container was discoloured. The product was immediately stirred and it was found that material in the centre of the mixture had blackened due to charring.

The product was removed from the container, thoroughly mixed, and analyzed. The analysis of the partially charred product was:

| | |
|---|---|
| Propionate acid content | 75.7% |
| Free moisture | 1.7% |

It was determined, by conducting a mass balance, that 13.8% of the initial propionic acid had been lost during the trial. In addition 3.3% of the calcium had been lost.

### EXAMPLE 2

### Calcium Propanoate

The procedure of Example 1, was followed, but the propionic acid was first diluted with water to 85% m/m prior to charging the reaction chamber.

The rate of temperature increase in the magma was substantially lower and a slower evolution of steam occurred. However the batches were still completed every 30 seconds. At the end of the addition phase, the magma temperature had risen to 108° C. The temperature peaked at 125° C., after approximately 11 minutes. The magma dried and formed a mixture of flakes and a hard cake approximately 16 minutes after the commencement of the trial and there was no evidence of charring. The product was broken up manually, and sieved without drying. The screened product was thoroughly mixed, re-weighed and analyzed. The analysis of the product was:

| | |
|---|---|
| Propionate acid content | 76.0% |
| Calcium | 19.1% |
| Free moisture | 5.1% |

It was determined, by conducting a mass balance, that 7.3% of the initial propionic acid had been lost during the trial. In addition 1.4% of the calcium had also been lost. Some of this product was dried further and analyzed. The product comprised

Propionate acid content 79.0%

| | |
|---|---|
| Calcium | 20.0% |
| Free moisture | 1.1% |

This example shows how charring and acid loss is reduced by pre-diluting the propanoic acid.

### EXAMPLE 3

### Calcium Propanoate

The procedure of Example 1 was followed but the propionic acid was diluted to 97% m/m, by charging water and propionic acid separately into the reaction chamber, and the calcium oxide was replaced with a mixture of calcium oxide and calcium carbonate in a 1:1 molar ratio.

The rate of temperature increase in the magma was again substantially lower and a slower evolution of steam occurred. However the batches were still completed every 30 seconds. By the end of the addition phase the magma temperature had risen to 109° C. The temperature peaked at 115° C. after approximately 15 minutes. The magma dried and formed a mixture of flakes and a hard cake approximately 25 minutes after the commencement of the trial and there was no evidence of charring. The product was broken up manually, and sieved without drying. The screened product was thoroughly mixed, re-weighed and analyzed.

The analysis of the product was:

| | |
|---|---|
| Propionate acid content | 73.6% |
| Calcium | 20.8% |
| Free moisture | 2.8% |

The losses of propionic acid and calcium were 5.2% and 3.3% (of the initial quantities charged), respectively. This example shows how both pre-dilution and the use of a less reactive base reduces charring and acid loss.

### EXAMPLE 4

### Calcium Magnesium Acetate

Acetic acid 98.5% (165.16 g), water (15.60 g), calcium oxide 96.5% (27.00 g) and magnesium oxide 86% (43.00 g) were weighed out. The water and acetic acid were mixed together and the solids blended together. The components were then each divided into two equal batches prior to mixing.

The first aqueous acid solution was added to a pre-weighed mixing vessel over a period of approximately 3 seconds. The first calcium/magnesium oxide sample was then added to the acid solution over a period of approximately 6 seconds, whilst agitating vigorously. The reaction started almost immediately, and agitation was continued for a further 5 seconds. The material was then tipped out of the container into a reaction vessel over a period of five seconds. The second aqueous acid solution was added to the mixing vessel and the same procedure was followed. The total time (addition, mixing and discharge) was about 22 seconds. The reaction was allowed to go to completion.

After milling and screening, the analysis of the product was as follows:

| | |
|---|---|
| Acetic acid | 71.60% |
| Calcium | 8.67% |
| Magnesium | 10.12% |
| Moisture | 6.86% |

### EXAMPLE 5

### Calcium Citrate

Citric acid monohydrate (208,55 g) was dissolved in water (50,0 g) at 85° C. to produce a 73.8% solution and the heat was removed. Three samples of calcium oxide (20.98 g, 20.99 g and 20.98 g) were weighed out and three samples of the citric acid solution (63.15 g, 63.14 g and 63.18 g) were weighed out at 74° C. The first calcium oxide sample was added to the first citric acid sample whilst agitating vigorously.

The agitation was continued for a period of approximately 5 seconds, and the mixture was emptied into a separate receiving container for reaction to complete. During the mixing period the temperature of the solution rose rapidly, reaching boiling point before the mixture was discharged. The same process was repeated successively with the remaining samples of citric acid and calcium oxide. The reacting mixture remained in the receiving container without any further agitation and the temperature of the mixture rose to 110° C.

Approximately 6 minutes after the calcium oxide had been added to the first citric acid solution, the resulting product could be tipped out of the reaction container. The product was friable, with a slight smell of quicklime. The product was crushed and screened through a 1.18-mm screen prior to analysis. The analysis of the product was:

| | |
|---|---|
| Citrate content | 66.6% |
| Calcium content | 22.5% |
| Free moisture | 3.7% |

### EXAMPLE 6

### Potassium Acetate

Commercial potassium hydroxide flakes were milled rapidly to produce a granular material with a particle size of 95% <1.18-mm. Three samples of 98.5% acetic acid (62.1 g each) and three samples of the milled potassium hydroxide (57.7 g each) were weighed out.

The first sample of the potassium hydroxide was added to the first sample of acetic acid in a mixing vessel with vigorous agitation over a period of about 8 seconds. The temperature of the solution increased rapidly and about 3 seconds after the completion of the hydroxide addition, the entire effervescing solution was discharged into a receiving vessel and left to react. The time between adding the hydroxide to the acid solution and emptying the mixing vessel was approximately 17 seconds. The process was repeated twice more with the second and third samples of acid and hydroxide.

The product reacted rapidly and produced a strong smell of acetic acid. Part of the steam plume condensed in the area immediately around the mixing area. The product analysed as:

| | |
|---|---|
| Potassium | 58.8% |
| Calcium | 33.2% |
| Moisture | 4.5% |

### EXAMPLE 7

The method of Example 1 was repeated using formic acid, acetic acid and water. The analysis of the products calcium formate and calcium acetate was:

| | Calcium formate | Calcium acetate |
|---|---|---|
| % formate | 65.4 | — |
| % acetate | — | 71.8 |
| % calcium | 34.9 | 23.2 |
| % water | 1.3 | 2.3 |

### EXAMPLE 8

The methods of Example 2, 3, 4, 5 and 6 were repeated but the magma, after transfer from the initial mixing vessel to the receiving vessel was further agitated to encourage the liberation of steam and other vapours and to aid in drying the reaction mixture.

### EXAMPLE 9

### Totally Enclosed Batch Process

Referring to the drawings, reference numeral 10 generally indicates an installation for the continuous production of a salt of a C₁-C₁₀ acid. The installation 10 includes a carousel 12 which rotates in the direction of the arrows 14. Twelve hoppers 16.1-16.12 are mounted on the carousel 12.

The installation 10 includes a base silo 18 and a storage tank 20 which holds a preheated aqueous solution of a C₁-C₁₀ acid. Feed lines 22, 24 extend from the silo 18 and the tank 20 to a mixing installation 26 which includes a reaction/mixing vessel (not shown) and a measuring device (not shown) for measuring the volume or mass of the base and the aqueous acid. A feed line 28 extends from the mixing installation 26 for feeding the reaction mixture into the receiving hoppers 16.1-16.12.

FIGS. 2 and 3 show the hopper 16.12 and a part of the carousel 12 in further detail. The carousel 12 includes a rotatable support platform 30 on which the hoppers 16.1-16.12 are mounted on pivot mechanisms generally indicated by reference numeral 32 so that each hopper 16.1-16.12 can be tilted in the direction of the arrow 34 as shown in FIG. 3. A hydraulic arm, (not shown) operates to tip the hoppers 16.1-16.12 so that the reaction mixture can be discharged from the hoppers 16.1-16.12. The installation 10 further includes a spillage plate 56.

The carousel 12 and the hoppers 16.1-16.12 are enclosed in an annular tunnel structure 40 (FIGS. 2 and 3) which is provided with an extractor 42 (FIG. 1). The tunnel structure 40 is provided with a door 46 which opens in the direction of the arrow 48 by a hydraulic ram mechanisms 50 when a hopper 16.1-16.12 is tipped as shown in FIG. 3. When the hopper 16.1-16.12 returns to its upright position on the carousel 12, the door 46 closes again in the direction of the arrow 52 (FIG. 3). Material 51 tipped from the hopper 16.1-16.12 is optionally passed through a crumbler (not shown) and falls onto a conveyor belt 54 from where it is conveyed to a receiving vessel.

### EXAMPLE 10

### Open Process

Referring now to FIG. 4, reference numeral 80 generally indicates another embodiment of an installation for the production of a salt of a C₁-C₁₀ acid. The installation 80 includes a pair of reaction vessels or bins 82, 84 which are pivotally mounted within a housing, generally indicated by reference numeral 86. A part of the housing 86 forms a roof 88 above the bin 82 and a roof 90 above the bin 84. A feed conduit 92 extends from a reaction vessel 96. The conduit 92 can swivel in the direction of the arrow 94 so that it can feed reaction mixture from the reaction vessel 96 through the roofs 88, 90 into the bins 82, 84. Extraction conduits 97, 98 extend from the roofs 88, 90 to a common extraction conduit 100.

The bins 82, 84 are pivotally mounted so that they can be tipped (the bin 82 being depicted in its tipped position). Stop formations 102, 103 are provided below the bins 82, 84 and are positioned so that impact of the bin 82, 84 on the stop formation 102, 104 will serve to dislodge material in the bin 82, 84. Material 104 tipped from the bins 82, 84 falls onto a conveyor 106. In another embodiment of the invention (not shown), the stop formations 102, 104 are replaced with cam mechanisms which jolt the bins to dislodge the contents so that an entire load (up to 2 tons of material) is not dislodged in a single mass.

In the embodiment depicted in the drawings the bins are lined with polytetrafluoroethylene or a material having similar properties and have a capacity of about 3 m². The bins 82, 84 are approximately 1,9 m in diameter with a cylindrical section of about 0,5 m in length and a 1,9 m diameter hemispherical bottom. In the filling position, as shown for bin 84, the bin 84 is substantially flush against the roof 90 above it. In another embodiment of the invention (not shown) the reactants are fed into the bin 84 and fumes are removed from the bin 84 via a common conduit. The installation 80 also includes mechanisms (not shown) for tipping the bins 82, 84 and for returning them to their upright positions. The installation 80 further includes valves (not shown) in the extraction conduits 96, 98 which are closed by the programmable logic controller (PLC) when the tipping mechanism is activated to prevent air being pulled through the relevant side of the extraction system. Each valve is reopened when the mechanism returns the bin 82, 84 to its upright position. In this embodiment of the invention, the PLC ensures that one bin 82, 84 is always in the filling position, thus minimizing risk of damage to the extraction fan. The surge bin itself is partially closed off for example with small suctions in the roof (not shown) or via the enclosed belt conveyor.

The bins 82, 84 are fed via a revolving chute (not shown) under the exit of the reaction vessel 96. The chute swivels to feed bin 82 or 84 as required. This embodiment of the invention does not require a valve at the bin 82, 84 as the reactant flow is controlled from the reaction vessel and material only flows down the chute when the bin 82, 84 is in its upright position. The inlet not in use permits air to be sucked through from the outside thereby minimizing odours in the general area.

This particular embodiment of the invention is relatively inexpensive. There are few moving parts, no operator exposure and the entire installation is enclosed.

### Discussion

There are effectively three ways in which the reaction can be carried out. These are referred to below as the “open process”, the “totally enclosed batch process” and the “enclosed continuous process”. All three processes are based on controlling the amount of acid in the form of an aqueous solution.

### The Open Process

The open process is a continual batching operation. In this process, the combination of the raw materials and the frequency of batches is fast enough to match that of a continuous process even though each raw material batch is weighed individually. As acid solution is drawn from the tank for use in the process, it is replenished by fresh acid solution.

The success of the process is determined by rapid and continuous batching of the raw materials, rapid mixing of the raw materials and rapid transfer of the reacting reaction mixture, or magma.

Material Dosing

In the dosing stage each of the raw materials i.e. water, the base and the aqueous solution of the acid are measured separately but simultaneously and precise quantities are discharged sequentially into the mixing vessel. The operation is controlled by a programmable logic controller (or PLC) to ensure repeatability and accuracy of both the quantities and time intervals involved.

As soon as the acid or water tanks or the hopper which form parts of the mixing installation have been emptied, the PLC initiates the weighing sequence and refills the vessels with the correct quantities of material from storage. Thus, by the time the reaction mixture is ready for discharge from the reactor, the raw materials for the next batch have already been weighed out.

Mixing

The raw materials are discharged in sequence into the reactor with the acid discharged first followed by any formulation water. As soon as the valve opens to discharge the acid, the agitator starts. Once the acid valve has opened and the agitator has started, the PLC will dispense the water and open the base discharge valve. After a preset interval, compressed air is blown into the base weighing vessel to force the base out. Thus the acid, water and base are discharged almost simultaneously into the mixing zone created by the rotating agitator.

Mixing is continued for a predetermined time. The time is determined by how rapidly the reaction mixture begins to boil. The mixing time can be longer in the case of a coarse or lower reactivity base or where the acid is diluted.

The mixer is linked to a fume extraction system to remove steam and fumes produced during the reaction.

The Rapid Transfer Stage

Initially, a liquid mixture comprising free acid, base, water and some salt is present in the reactor. If this mixture is left in the reactor, the temperature rises rapidly, and the reaction mixture or magma starts to lose water and becomes extremely plastic and sticky. Ultimately this material would adhere to the mixing blades and the sidewall and transferring the material would be very difficult.

Before this happens, and after a preset time, the PLC opens the discharge valve of the reactor so that the reaction mixture is rapidly transferred into an adjacent unstirred receiving/reaction zone or den. The object of the rapid mixing step and rapid transfer is to initiate but not to complete the reaction between the acid and the unslaked lime so that the reaction is completed in a separate reaction zone.

After a preset time, the discharge valve on the reactor closes, and the entire cycle begins again. However, during the mixing and transfer periods, the acid, water and base weighing vessels have been refilled with the desired quantity of raw materials. There is accordingly no significant time delay waiting for the weighing stages to be completed. The reacting magma usually takes approximately 2-60 seconds to discharge.

Final Reaction Stage

In the reaction zone, the reaction is allowed to go to completion. However in order to fully utilize the heat of reaction between the acid and the base, several batches are dropped sequentially into the same reaction zone or den. Most of the steam and fumes arising from the reaction emanates in this zone and accordingly every reaction zone and den is fitted with suitable fume and steam extraction equipment.

Once a den, or reaction zone, is full the reactor outlet swivels and commences discharging into a second (or third) adjacent den. The same sequence is followed until the second den is full. Whilst one den is filling up, the other(s) are matured or emptied. Generally the material remains in the static den for a period ranging from 3 to 60 minutes.

The material removed from the den is generally transported to a bay to mature and cool, or taken to further processing stages such as granulation, enrichment, cooling and dressing prior to packaging.

Drying

It has been found that the initial product discharged from the dens has a moisture-content of about up to 8%. If this product is left to mature and cool in a bay, the mixture content drops to below 2,5% over a period of about 24 hours.

Continued Agitation After Rapid Transfer

Instead of being maintained static in the receiving zone/den, the reaction mixture may be subjected to continued agitation. Such continued agitation facilitates drying by the liberation of steam from the reaction mixture.

### The Totally Enclosed Batch Process

There are several differences between the “open process” and the “totally enclosed batch process”.

In the “open process” the reacting magma is discharged into one or more static or moveable reaction zones or dens. These dens are fitted with fume extraction apparatus to remove the fumes and steam from the reaction area.

In the “totally enclosed batch process”, there are a number of reaction dens on a carousel as described under Example 8. However all the dens are located within a single totally enclosed fume and steam extraction system. The raw material weighing system and reactor are usually separated from and positioned above position number 1 (i.e. hopper 16.1) of the carousel. However fumes and steam arising in the weighers or in the reactor are also withdrawn to the fume extraction system.

Material is weighed out and discharged in a manner similar to the batch system. The charging time of the den is very similar to that of the “open process”. Once a den has reached the desired level the carousel is rotated to position another den/hopper under the reactor discharge. The process is then repeated. Once the second den is filled, the carousel rotates again to position a fresh den under the reactor discharge.

The filled dens rotate on the carousel, with the reaction continuing within each den. Effectively the static dens of the “open process” are replaced with rotating bins.

The number of dens on the carousel is selected so that the reaction has time to proceed to completion before the den needs to be emptied and refilled. The larger the number of dens, the longer the period prior to refilling, and the greater the flexibility in modifying the plant throughput.

At the end of the cycle, the full den reaches the last position on the carousel before refilling. At this point a hydraulic arm tips the den, discharging the contents into a lined bin. The lined bin optionally contains a rotary cutter to break up any lumps formed.

However the tipping mechanism and top of the bin remain enclosed within the fume control system. The bin discharges the contents onto an enclosed handling system, such as a shrouded belt conveyor. This handling system takes the product to a maturation heap or for further processing.

Each den and the discharge bin are made from steel or a suitable composite material. There are no sharp corners within the dens or bin, and each vessel is lined with a suitable, slippery, temperature-resistant material, such as PTFE or similar. The lining is selected to withstand the maximum temperature reached during the reaction, but still release the product easily on tipping.

The den size is designed typically to take 500-1000 kg of the product and there are typically about 12-16 dens or bins on the carousel. However, the size and number can be adjusted according to the capacity of the plant.

In an embodiment of the invention, the reactor is fed by appropriate continuous handling/weighing systems. The material is accumulated in the reactor and discharged before the product thickens significantly. In another embodiment a continuous high-speed mixer with a short retention time is employed. A flap eliminates spillage during the rotation of the dens.

### Enclosed Continuous Process

In the “enclosed continuous process” the same principles are followed as in both the open and the totally enclosed batch processes. As before, the key principles are: rapid weighing of the raw materials, rapid mixing, and rapid transfer to a reaction zone.

In different embodiments of the enclosed continuous process, the raw materials are weighed out either on a batch basis, as described for the totally enclosed batch process above, or on a continuous basis using continuous mass measuring apparatus and a high-speed continuous mixer with a short residence time.

Effectively a moving den in the form of an enclosed endless belt replaces the static or moving dens of the batch processes. The enclosed belt is fitted with extraction ports to extract steam and fumes to a fume handling system.

The enclosed continuous process is initiated by weighing the raw materials and discharging the weighed materials sequentially into the reactor as described in the previous examples. The charging time of the reactor is essentially the same as that of the batch processes described above. Again, in different embodiments, the reactor is discharged batch-wise or continuously. The mixing time in the reactor is also essentially the same as that of the batch processes described above.

The reacting magma flows down a chute onto a suitable temperature-resistant endless flexible belt which is typically of a material such as thin stainless steel or PTFE. The belt speed can be varied to control the depth of material on the belt. In different embodiments, the belt is troughed (forming sidewalls and individual compartments) or folded on itself in the shape of a shallow “U”. The belt slopes slightly away from the feed end towards the product discharge end so that reaction mixture or magma flows away from the feed end as long as it is still fluid.

The walls of the belt (or pockets of the troughed conveyor) form the bottom and sidewalls of the moving belt, eliminating spillage. A roof with at least one steam/fume extraction point runs the entire length of the conveyor so that steam and fumes produced during the reaction are removed to the fume handling system.

The reaction between the acid and base proceeds essentially to completion on the belt. At a distance down the belt (determined by the plant capacity and speed of the belt) the material thickens, and acts as a dam wall against the further flow of fluid reacting magma.

At a point past the area where the material has hardened (nearer to the product discharge end), the belt flattens out. This is achieved by the belt running over a flat sloping plate or wide conveyor idler.

The flattening out of the product on the belt causes the heap of material travelling on the belt to pull apart, so that cracks and fissures are formed. The cracking up and crumbling of the surface allows steam and trapped fumes to escape. As this is taking place within the fume extraction hood, no fumes escape to the surrounding working area. The flattening out of the belt also results in the belt floor widening. This causes material to be dislodged from the sidewalls as they flatten out and reduces the chance of material adhering to the fume hood. At the end of the belt, the material falls into a lined bin equipped with a rotary cutter and rotating brushes clean the belt as it returns to the feed end. The material freed by the brushes drops into the bin. The top of the bin is enclosed within the fume extraction hood.

The bin discharges onto a suitable enclosed transport system, which moves the product to a maturation heap or for further processing.

Typically the product depth or thickness on the belt will be around 5-90 cm across a belt up to 2 m wide. The enclosed system is typically up to 25 m long with a roof height of up to 2 m. The material remains on the belt for a period of from 3 to 120 minutes, depending on several factors such as plant throughput and base reactivity.

It is an advantage of the invention that the problems associated with the “plastic” stage of prior art processes known to the Applicant are avoided by the method of the invention.

Raw material costs are also reduced by using oxides or oxides and carbonates as the base instead of the carbonates or hydroxides which have been used in prior art procedures. The method of the invention also reduces the risk of charring and uses less power than prior art processes which require heavy duty mixers, blenders and the like. Losses can also be relatively easily controlled and far less drying of the product is required than in the case of prior art processes. This results in substantial cost saving.

The Applicant believes that the method of the invention will result in the cheaper and more efficient production of the salts of C₁-C₁₀ acids than those described in the prior art. In particular, the method of the invention avoids the problems associated with handling a reaction product when it is in its “plastic” phase because the reaction mixture is transferred before this stage is reached. It also reduces acid loss and eliminates charring by incorporating water which prevents overheating. The product also requires less drying than the products of prior art processes known to the inventor and accordingly is more energy efficient.

1. A method of making the salt of an acid selected from C₁-C₁₀ carboxylic acids, aromatic carboxylic acids of the formula Ph-(CH₂)ₓ—CO₂H where x is 0-4, and glycerophosphoric acid, the method including the steps of
combining and mixing the acid and a base selected from the oxides, hydroxides and carbonates of sodium, potassium, calcium and magnesium, or a mixture of any two or more thereof, in a first reaction zone, the combining and mixing step being carried out over a first period to produce a reaction mixture in the first reaction zone;
transferring the reaction mixture at the end of the first period from the first reaction zone to a second reaction zone the transferring step being carried out over a second period; and
allowing heat generated by reaction between the acid and the base in the second reaction zone to drive off sufficient water to produce a product mixture containing less than about 8% (m/m) water,
provided that the acid is not 2-hydroxy-4-thiomethylbutyric acid.

2. A method as claimed in claim, in which the first period is 3-180 seconds and the second period is 2-60 seconds.

3. A method as claimed in claim 1, in which the combining step takes about 2-60 seconds.

4. A method as claimed in claim 2, in which the first period is 3-60 seconds.

5. A method as claimed in any one of claims 2 to 4 inclusive, in which the second period is 3-30 seconds.

6. A method as claimed in any one of the preceding claims, in which the carboxylic acid is a substituted or an unsubstituted C₁-C₁₀ acid.

7. A method as claimed in any one of claims 1 to 6 inclusive, in which carboxylic acid is monocarboxylic acid selected from formic acid, acetic acid, propanoic acid, butanoic acid, pentanoic acid, hexanoic acid, heptanoic acid, octanoic acid, nonanoic acid, decanoic acid and their alkylated or hydroxylated analogues.

8. A method as claimed in any one of claims 1 to 6 inclusive, in which the carboxylic acid is a dicarboxylic acid selected from oxalic acid, malonic acid, succinic acid, glutaric acid, adipic acid, pimelic acid, suberic acid, azelaic acid, sibaccic acid and their unsaturated, alkylated or hydroxylated analogues.

9. A method as claimed in any one of claims 1 to 5 inclusive, in which the acid is benzoic acid or phenylacetic acid.

10. A method as claimed in any one of the preceding claims, in which the acid is in the form of an aqueous solution.

11. A method as claimed in claim 10, in which the aqueous solution has a concentration of about 60-99,5% by mass of the acid.

12. A method as claimed in any one of the preceding claims, which includes allowing the heat generated to drive off sufficient water to produce a product mixture containing less than about 2,5% water.

13. A method as claimed in any one of claims 10 to 12 inclusive, which includes the prior step of warming the solution of acid to 50-96° C.

14. A method as claimed in any one of the preceding claims, which includes the further steps of successively combining and mixing a plurality of batches of the acid and base with water in the first reaction zone to produce successive batches of the reaction mixture and successively transferring each of the batches to the same second reaction zone.

15. A method as claimed in any one of the preceding claims which includes agitating the reaction mixture in the second reaction zone.

16. A method of making salt of an acid selected from C₁-C₁₀ carboxylic acids, aromatic carboxylic acids of the formula Ph-(CH₂)ₓ—CO₂H where x is 0-4, benzoic acid, and glycerophosphoric acid, the method including the steps of
combining and mixing the acid, a base selected from the oxides, hydroxides and carbonates of sodium, potassium, calcium and magnesium, or a mixture of any two or more thereof and water in a first reaction zone to produce a reaction mixture in the first reaction zone;
continuously transferring the reaction mixture from the first reaction zone to a second reaction zone, the reactants being added to the first reaction zone in successive batches and the reaction mixture being continuously removed from the first reaction zone at a rate which is selected so that the residence time of the reaction mixture in the first reaction zone is between about 1 and 180 seconds; and
allowing heat generated by reaction between the acid and the base in the second reaction zone to drive off sufficient water to produce a product mixture containing less than about 8% water,
provided that the acid is not 2-hydroxy-4-thiomethylbutyric acid.

17. A method as claimed in claim 16 which the heat generated is allowed to drive off sufficient water to produce a product mixture containing less than about 2,5% water.

18. A continuous method of making the salt of an acid selected from C₁-C₁₀ carboxylic acids, aromatic carboxylic acids of the formula Ph-(CH₂)ₓ—CO₂H where x is 0-4, and glycerophosphoric acid, the method including the steps of
simultaneously feeding, into a reaction zone, an aqueous solution of the acid, and a base selected from the oxides, hydroxides and carbonates of sodium, potassium, calcium, magnesium or a mixture of any two or more thereof to produce a reaction mixture in the reaction zone; and
transferring the reaction mixture from the reaction zone to a second zone at a rate which is selected so that the residence time of the reaction mixture in the reaction zone is sufficient to allow heat generated by reaction between the acid and the base in the reaction zone to drive off sufficient water to produce a product mixture containing less than about 8% water,
provided that the acid is not 2-hydroxy-4-thiomethylbutyric acid.

19. A method as claimed in claim 18, in which the rate is selected so that the residence time of the reaction mixture in the reaction zone is 3-20 minutes.

20. A continuous method of making a salt of an acid selected from C₁-C₁₀ carboxylic acids, aromatic carboxylic acids of the formula Ph-(CH₂)ₓ—CO₂H where x is 0-4, and glycerophosphoric acid, the method including the steps of
simultaneously feeding, into a reaction zone, an aqueous solution of the acid, and a base selected from the oxides, hydroxides and carbonates of sodium, potassium, calcium and magnesium or a mixture of any two or more thereof to produce a reaction mixture in the reaction zone; and
transferring the reaction mixture from the reaction zone to a second zone at a rate which is selected so that the residence time of the reaction mixture in the reaction zone is sufficient to initiate reaction between the acid and the base but not sufficient to drive off water from the reaction mixture and allowing heat generated by further reaction between the acid and the base in the second zone to drive off sufficient water to produce a product mixture containing less than about 8% water, provided that the acid is not 2-hydroxy-4-thiomethylbutyric acid.

21. A calcium, magnesium, sodium or potassium salt of a C₁-C₁₀ carboxylic acid, aromatic carboxylic acid of the formula Ph-(CH₂)ₓ—CO₂H where x is 0-4 or of glycerophosphoric acid prepared by a method as claimed in any one of claims 1 to 20 inclusive.

22. A method as claimed in claim 1 or claim 16 or claim 18 or claim 20, substantially as herein described and illustrated.

23. A salt as claimed in claim 21, substantially as herein described and illustrated.

24. A new method of making the salt of an acid or a new salt of acid, substantially as herein described.
